(19) United States
(12) Patent Application Publication
(10) Pub. No.: US 2009/0326617 A1
(43) Pub. Date: Dec. 31, 2009

(54) LASER TREATMENT DEVICE

(75) Inventor: Hideki Asano, Ashigarakami-gun (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM CORPORATION, Tokyo (JP)

(21) Appl. No.: 12/482,518

(22) Filed: Jun. 11, 2009

(30) Foreign Application Priority Data
Jun. 27, 2008 (JP) 2008-168976

(51) Int. Cl. A61N 5/06

(52) U.S. Cl. 607/89

(57) ABSTRACT

There is provided a laser treatment device including: an image acquisition section that acquires an image of a treatment region including a lipid component inside a living body and surroundings of the treatment region; a position acquisition section that acquires a position of the treatment region in an optical axis direction of laser light, on the basis of image data of the image acquired by the image acquisition section; an irradiation section that irradiates laser light with a wavelength from 1201 nm to 1227 nm at the treatment region from outside the body; and a focusing section that focuses the laser light irradiated from the irradiation section at the position of the treatment region acquired by the position acquisition section.

### CROSS-REFERENCE TO RELATED APPLICATION

This application is based on and claims priority under 35 USC 119 from Japanese Patent Application No. 2008-168976 filed on Jun. 27, 2008.

### BACKGROUND

1. Technical Field

The present invention relates to a laser treatment device, and more particularly to a laser treatment device for irradiating laser light at and treating lipid components in a body.

2. Related Art

In general, a major cause of arteriosclerosis is excess lipid components accumulating in blood vessels and forming plaques, and the plaques that are formed narrowing the blood vessels.

Currently, the three methods presented below are used as the main methods of treatment of arteriosclerosis.

Method 1: Cutting into an arteriosclerosis region by surgery and directly removing blood vessel inner walls at which plaques are present.

Method 2: Inserting a stent into a blood vessel and stretching the blood vessel.

Method 3: Inserting a laser light irradiation unit into a blood vessel, directly irradiating strong laser light onto a plaque, and vaporizing and removing the plaque.

Method 1 is highly invasive and there is a large burden on the body of the patient in surgery. Moreover, depending on the region at which the arteriosclerosis has occurred, complications such as infarctions and the like may result. At the neck region in particular, there is a high possibility of a stroke resulting. As for Method 2, plaques will re-occur in the vicinity of the stent, so this is generally a temporary measure.

As Method 3, a technique is known (see, for example, Japanese Patent Application Laid-Open (JP-A) No. 2005-237827) of inserting a catheter into a blood vessel and irradiating laser light through an optical fibre to a treatment area. A further technique is known (see, for example, JP-A No. 2007-029277) of irradiating laser light at a treatment region from the probe part of an ultrasound probe that is inserted into a body cavity. These are less invasive than Method 1 and there is little re-occurrence of plaques.

However, with Method 3, similarly to Method 1, there is a high possibility of a stroke resulting, and there is a possibility of inadvertent damage to blood vessels.

Therefore, treatment of plaques within the body from outside the body is desired. However, when laser light is irradiated from outside the body using, for example, the ultrasonic probe mentioned in JP-A No. 2007-029277, inflammation, pain and the like may occur at the skin surface (body surface).

### SUMMARY

An object of the present invention is to provide a laser treatment device capable of irradiating a laser at a lipid component within a living body from outside the body.

In order to achieve the object described above, the present invention provides a laser treatment device including:

an image acquisition section that acquires an image of a treatment region including a lipid component inside a living body and surroundings of the treatment region;

a position acquisition section that acquires a position of the treatment region in an optical axis direction of laser light, on the basis of image data of the image acquired by the image acquisition section;

an irradiation section that irradiates laser light with a wavelength from 1201 nm to 1227 nm at the treatment region from outside the body; and

a focusing section that focuses the laser light irradiated from the irradiation section at the position of the treatment region acquired by the position acquisition section.

The position acquisition section acquires the position of the treatment region in the optical axis direction of the laser light on the basis of the image data of the image, which is acquired by the image acquisition section, of the treatment region including the lipid component within the living body and the treatment region vicinity. The laser light with a wavelength from 1201 nm to 1227 nm, which is irradiated at the treatment region from outside the body by the irradiation section, is focused on the position of the treatment region, which is acquired by the position acquisition section, by the focusing section.

Thus, it is possible to irradiate the laser light, with a wavelength that has a higher coefficient of absorption in the lipid component than in water or skin or the like, at the treatment region within the living body from outside the body. Therefore, a laser may be irradiated at a lipid component inside the living body from outside the body and treatment performed.

The irradiation section may include a light intensity alteration section that alters a light intensity of the irradiated laser light, and the focusing section may include a focusing lens and a focusing distance variation section that alters a focusing distance of the focusing lens, and the laser treatment device may further includes a control section that controls the light intensity alteration section and the focusing distance variation section in accordance with the position of the treatment region such that the light intensity of the laser light is a light intensity capable of treating the treatment region.

The control section controls the light intensity of the laser light and the focusing distance of the focusing lens, by controlling the light intensity alteration section and the focusing distance variation section, on the basis of the position of the treatment region, such that the light intensity of the laser light is a light intensity capable of treating the treatment region. The meaning of the term “a light intensity capable of treating the treatment region” includes a light intensity of laser light that is capable of dissolving the lipid component included at the treatment region when irradiated at the treatment region inside the living body from outside the body and/or of causing apoptosis or necrosis of the lipid component.

Thus, laser light with a light intensity required for treatment may be irradiated at a more precise position. Therefore, the treatment may be carried out efficiently.

The focusing distance variation section may comprise a movement portion that moves the focusing lens in the optical axis direction of the laser light.

By the movement portion moving the focusing lens in the optical axis direction of the laser light, the focusing position of the laser light may be altered in the direction to the interior of the living body (an oscillation direction). Thus, the laser light may be more precisely irradiated at the treatment region.

The focusing lens may comprise a liquid lens whose focusing distance is alterable.

Because the focusing lens is a liquid lens whose focusing distance is alterable, the focusing position of the laser light may be altered in the direction to the interior of the living body (the oscillation direction). Thus, the laser light may be more precisely irradiated at the treatment region.

Further, plural the irradiation sections may be provided, and the focusing section may focus the plural laser light irradiated from the plurality of irradiation sections.

Because the irradiation section is plurally provided and the plural laser lights are focused at the treatment region, cases in which high power output (light intensity) is required can be dealt with.

The laser treatment device according to the present invention may further comprise a display section that displays the image of the treatment region and the surroundings of the treatment region acquired by the image acquisition section.

Because the image of the treatment region and the treatment region vicinity is displayed by the display section, a state of treatment of the treatment region may be more easily ascertained.

The laser treatment device according to the present invention may further comprise a cooling portion that cools a surface of the living body at which the laser light irradiated from the irradiation section is irradiated.

Because the surface of the living body at which the irradiated laser light is irradiated is cooled by the cooling portion, heating of the living body surface may be suppressed. Therefore, effects on the human body may be further ameliorated.

Further, the cooling section may include a pair of thin-film members that transmit the laser light, and a supply portion, between the pair of thin-film members, that supplies a liquid or gas for cooling the living body.

Because the liquid or gas for cooling the living body is provided by the supply portion between the pair of thin-film members, the surface of the living body may be cooled more optimally.

The image acquisition section may comprise an ultrasound image acquisition section that includes an ultrasound probe and that acquires an ultrasound image on the basis of reflected waves of ultrasound, which is outputted from the ultrasound probe toward the interior of the living body from outside the body.

Because the ultrasound image based on reflected waves of ultrasound is acquired by the ultrasound image acquisition section, the image of the treatment region and the treatment region vicinity may be acquired more precisely. Hence, the laser light may be more precisely irradiated only at the treatment region.

Further, a numerical aperture of the focusing section may be a numerical aperture such that a beam area of the laser light at a surface of the living body is at least 1.7 times a beam area of the laser light focused at the treatment region.

Damage to the surface of the living body may be better prevented by setting the numerical aperture of the focusing section such that the beam area of the laser light at the surface of the living body is at least 1.7 times the beam area of the laser light focused at the treatment region.

As described hereabove, according to the present invention, because laser light with a wavelength from 1201 nm to 1227 nm is irradiated toward the inside of a body from outside the body, an effect is provided in that a laser treatment device capable of treating lipid components inside a body from outside the body can be provided.

### BRIEF DESCRIPTION OF THE DRAWINGS

Exemplary embodiments of the present invention will be described in detail based on the following figures, wherein:

FIG. 1 is an explanatory diagram for explaining a wavelength of laser light that is irradiated by a laser treatment device relating to an exemplary embodiment of the present invention;

FIG. 2 is an explanatory diagram for explaining the wavelength of laser light that is irradiated by the laser treatment device relating to the exemplary embodiment of the present invention;

FIG. 3 is an explanatory view for describing a light intensity, of the laser light that is irradiated by the laser treatment device relating to the exemplary embodiment of the present invention, and a position of a plaque;

FIG. 4 is an explanatory view for describing a beam area, of the laser light that is irradiated by the laser treatment device relating to the exemplary embodiment of the present invention, and a numerical aperture;

FIG. 5 is an explanatory view for describing an example of a case in which a focusing lens, which focuses the laser light that is irradiated by the laser treatment device relating to the exemplary embodiment of the present invention, is a liquid lens;

FIG. 6 is a schematic structural view illustrating an example of general structure of a laser treatment device relating to a first exemplary embodiment of the present invention;

FIG. 7 is a schematic view of a portion of an example of an ultrasound probe relating to the first exemplary embodiment of the present invention;

FIG. 8 is a sectional view of the whole of the example of the ultrasound probe relating to the first exemplary embodiment of the present invention;

FIG. 9 is an explanatory view for describing an example of a treatment method with the laser treatment device relating to the first exemplary embodiment of the present invention;

FIG. 10A is a different explanatory view for describing the example of the treatment method with the laser treatment device relating to the first exemplary embodiment of the present invention;

FIG. 10B is a different explanatory view for describing the example of the treatment method with the laser treatment device relating to the first exemplary embodiment of the present invention;

FIG. 11 is a flowchart illustrating an example of a treatment method with the laser treatment device relating to the first exemplary embodiment of the present invention;

FIG. 12 is a schematic view of a portion of an example of an ultrasound probe relating to a second exemplary embodiment of the present invention; and

FIG. 13 is a sectional view of the whole of the example of the ultrasound probe relating to the second exemplary embodiment of the present invention.

### DETAILED DESCRIPTION

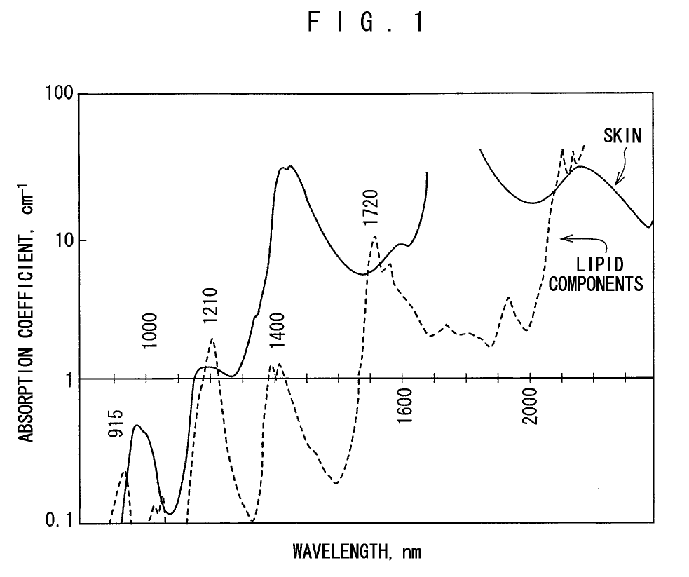

Firstly, an optical system of an exemplary embodiment of the present invention will be described in detail with reference to FIG. 1 to FIG. 5.

A laser treatment device of the exemplary embodiment of the present invention irradiates laser light toward the interior of a body from outside the body. When the laser light is irradiated inside the body from outside the body, it is desired that a coefficient of absorption of the laser light is higher in plaques than in blood or water or the like, and that the laser light is not absorbed at the body surface (the skin).

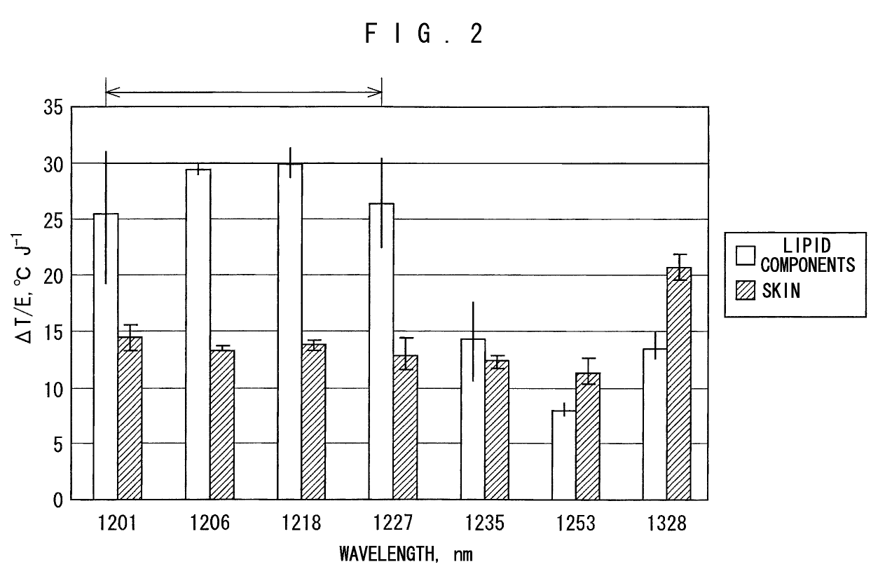

As shown in FIG. 1 and FIG. 2, laser light with a wavelength from 1201 nm to 1227 nm has a higher coefficient of absorption in lipid components (fats) than in water, and is effectively absorbed by lipid components (Reference Document: Lasers in Surgery and Medicine, Vol. 38, pp 913-919 (2006) “Selective Photothermolysis of Lipid-Rich Tissues: A Free Electron Laser Study”). Therefore, laser light with a wavelength from 1201 nm to 1227 nm, particularly of 1210 nm, selectively and preferentially heats up a temperature of fat relative to neighboring body tissues. Thus, by suitably controlling irradiation conditions, plaques alone may be selectively removed. In addition, only a little of the laser light with a wavelength from 1201 nm to 1227 nm is absorbed at the skin.

Therefore, the laser treatment device of the exemplary embodiment of the present invention, by irradiating a laser light with a wavelength from 1201 nm to 1227 nm, removes only plaques (lipid components) inside the body with laser light irradiated from outside the body.

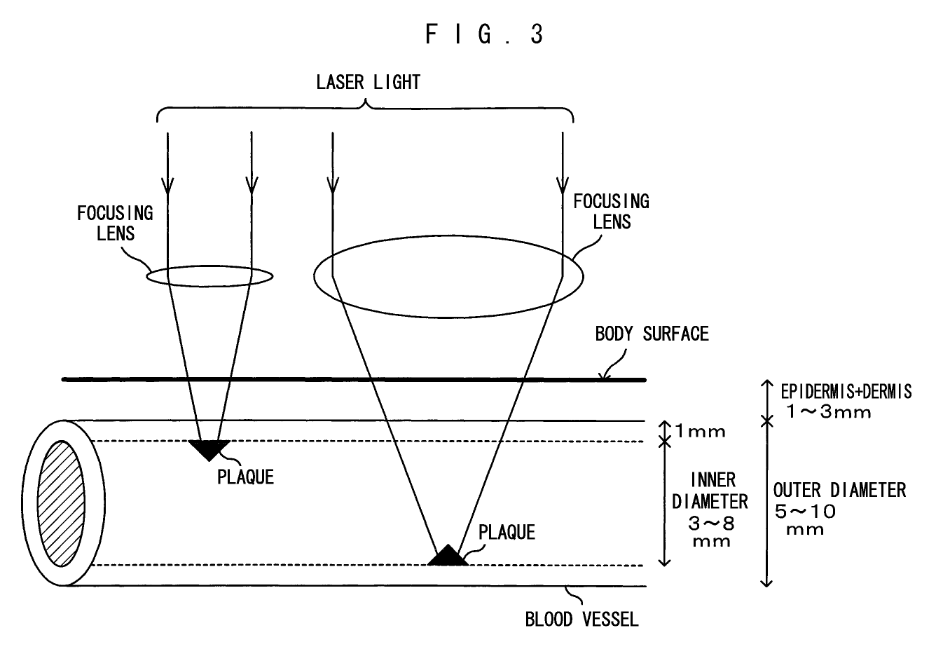

Further, as shown in FIG. 3, it is reckoned that the thickness of the epidermis and the dermis in the neck area is generally around 1 mm to 3 mm, the width of carotid arteries is about 10 mm for the relatively wide common carotid arteries and about 5 mm for the narrower internal carotid arteries, and the wall thickness of the blood vessels is about 1 mm. Therefore, plaques inside the blood vessels are disposed in a range of distances of about 2 mm to 12 mm from the surface of the skin (the body surface).

A radiation energy density required for dissolving lipid components, according to recent research, is about 75 J/cm², and it is reckoned that the temperature of body tissues rises to about 40° C. at such a time.

Given that a coefficient of absorption in water of laser light with a wavelength of 1210 nm is about 1.1 cm⁻¹, when a light intensity of laser light entering 2 mm to 12 mm into a body from the body surface is estimated, if an initial light intensity is I₀, the absorption coefficient is α and the laser light progression distance is x, the light intensity I after entry is calculated by I=I₀×10^{-αx}.

Therefore, the light intensity of the laser light after entering 2 mm to 12 mm into the body is attenuated to 60% to 5% of the light intensity before entering the skin (the body interior). That is, the deeper inside the body from the body surface the region of a plaque that is to be dissolved is, the higher the energy (light intensity) that is required. Therefore, it is necessary to consider damage to the skin.

Because laser light with a wavelength of 1210 nm has a larger coefficient of absorption in fat than in water, when the energy density irradiated at the skin is made smaller than 75 J/cm² as mentioned above, a temperature rise of the skin may be suppressed to below 40° C. Thus, damage to the skin such as a burn or the like may be prevented.

In order to reduce the energy density, it is preferable to increase the NA (numerical aperture) of the focusing lens.

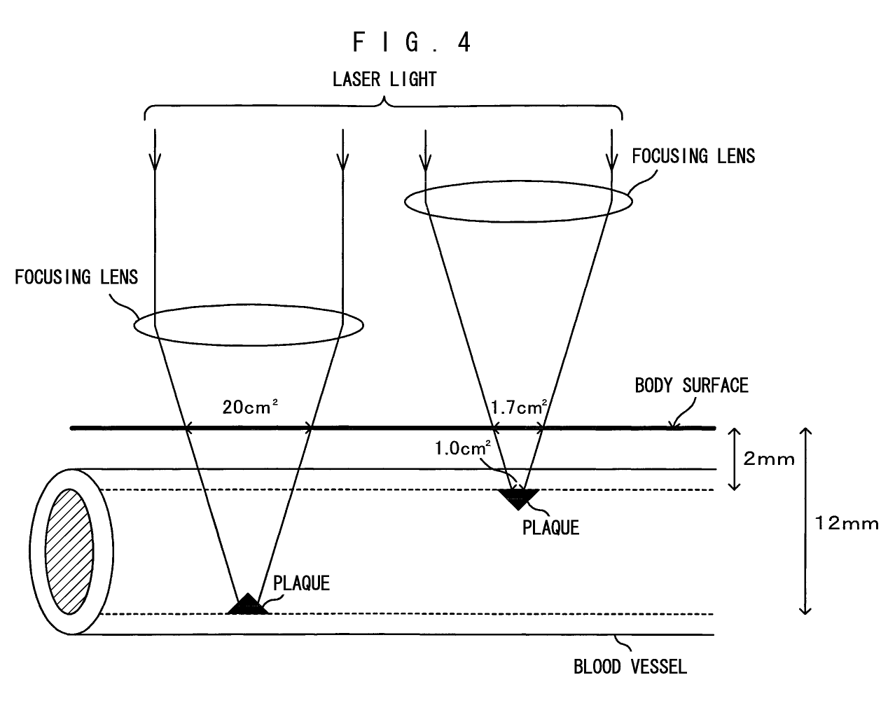

For example, as shown in FIG. 4, an optical system is used in which, if a beam area of laser light when focused at a plaque is 1 cm², the beam area of the laser light at the body surface is about 1.7 cm² or more in a case in which the plaque is at a depth of 2 mm from the body surface, and widens to about 20 cm² or more in a case in which the plaque is at a depth of 12 mm. That is, in order to irradiate laser light from outside the body and dissolve a plaque in a blood vessel, it is preferable that the beam area of laser light at the body surface be at least 1.7 times the beam area at the focused portion (the plaque). Further, in order to deal with plaques at any position of a carotid artery, it is preferable to design the laser treatment device such that the beam area widens to about 20 times or more.

A focusing lens with an NA (numerical aperture) to form such a design is employed. Converting this to a beam diameter of the laser beam, a beam diameter in order to produce 1.7 times the beam area corresponds to √1.7 times the beam diameter, and a beam diameter in order to produce 20 times the beam area corresponds to √20 times the beam diameter. When the NA of the focusing lens is estimated from this reckoning, if a lens whose refractive index n is 1.46 is employed, the NA, calculated from n×sin θ, is NA=0.11 to 0.21.

That is, it is more preferable to employ a lens with an NA of about 0.21 as the focusing lens, configure the focusing lens to be movable in an optical axis direction along a direction of depth from the body surface into the body, and alter power output of the laser light (light intensity) in accordance with a position of the lens.

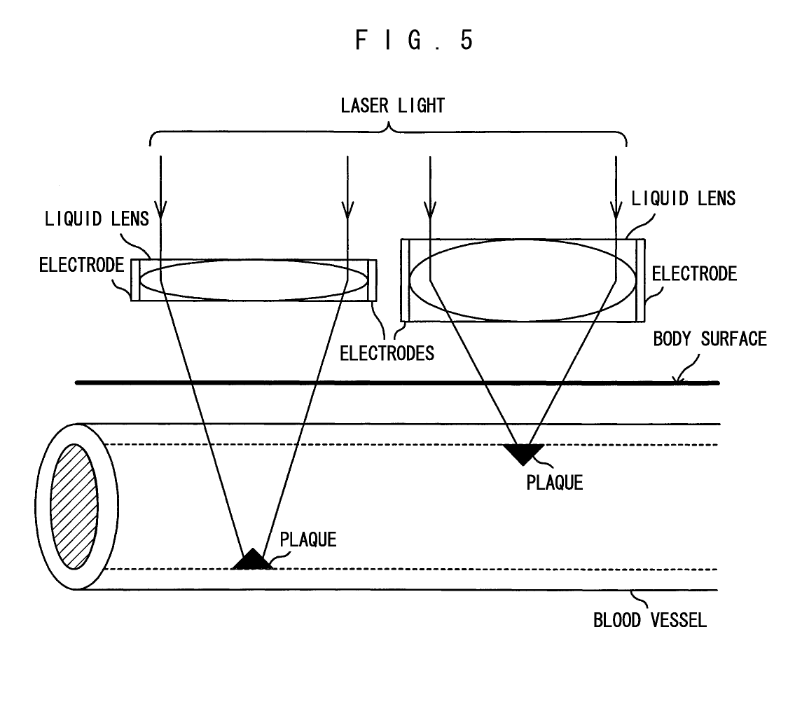

Alternatively, as shown in FIG. 5, a liquid lens whose focusing position is alterable may be employed as the focusing lens. The meaning of the term “liquid lens” includes a lens in which an aqueous liquid and an oil are sealed in a container, the inside of the container is partially subjected to a water-repellence treatment, and the shape of a boundary surface between the two liquids is altered and controlled by the application of voltages to electrodes at side faces of the container. When a liquid lens is employed, laser light may be safely irradiated at plaques by the laser light power output (light intensity) being altered in accordance with the focusing distance.

Next, a specific structure of the laser treatment device will be described in detail for subsequent exemplary embodiments.

### First Exemplary Embodiment

Herebelow, a specific example of an exemplary embodiment of the present invention is described in detail with reference to the drawings.

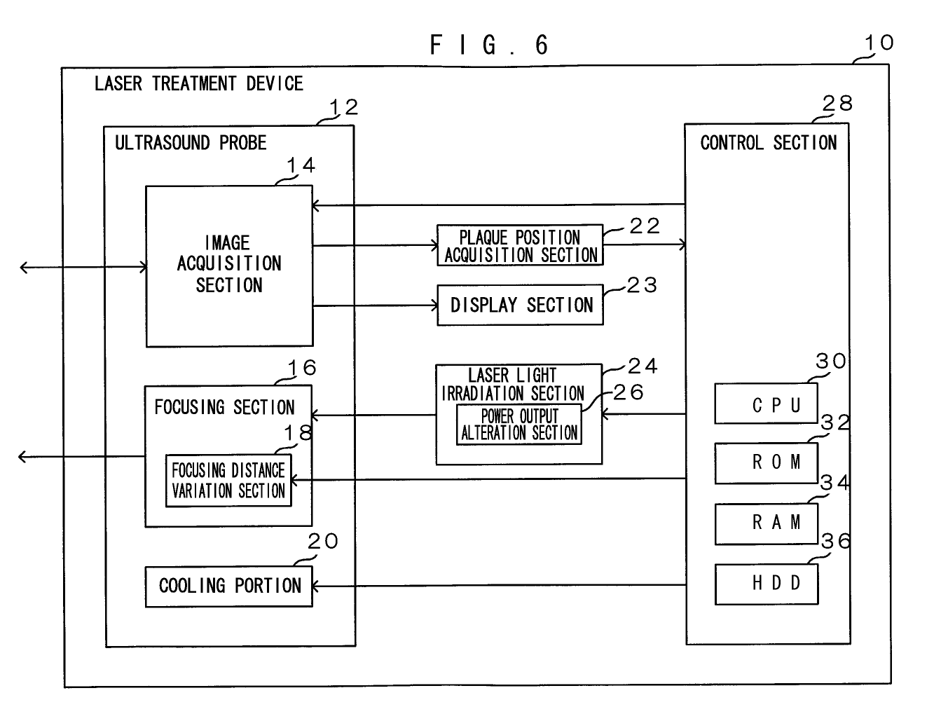

FIG. 6 is a structural view illustrating an example of general structure of a laser treatment device 10 of the present exemplary embodiment. The laser treatment device 10 of the present exemplary embodiment has a structure provided with an ultrasound probe 12, a plaque position acquisition section 22, a display section 23, a laser light irradiation section 24 and a control section 28.

The ultrasound probe 12 of the present exemplary embodiment is pressed against the body surface of a patient and an image of a plaque inside the body and surrounding portions is acquired, and the ultrasound probe 12 irradiates laser light. The ultrasound probe 12 is constituted to include an image acquisition section 14, a focusing section 16 including a focusing distance variation section 18, and a cooling portion 20 (which are described in more detail later).

On the basis of reflected waves of ultrasound outputted from the ultrasound probe 12, the plaque position acquisition section 22 of the present exemplary embodiment acquires a position, in depth and directions parallel to the body surface (directions orthogonal to the depth) or the like, to be a position of the plaque from the body surface. The plaque position acquisition section 22 calculates a progression distance of laser light to the plaque.

The display section 23 of the present exemplary embodiment is for displaying the image acquired by the image acquisition section 14. For example, a monitor, an LCD display or the like may be mentioned.

The laser light irradiation section 24 of the present exemplary embodiment is for irradiating laser light for treating plaques. The laser light irradiation section 24 is constituted to include a power output alteration section 26 for altering the intensity of the irradiated laser light.

The control section 28 of the present exemplary embodiment administers overall control of the laser treatment device 10. The control section 28 is constituted to include a CPU 30, a ROM 32, a RAM 34 and an HDD 36. The aforementioned formula of the relationship between the light intensity I after progress and the laser light progression distance x, a relationship between the laser light progression distance and the focusing distance of the focusing lens, and the like are stored in the HDD 36 beforehand. A processing flow for executing laser treatment processing (described in detail later) and parameters and the like are stored in the ROM 32. The processing flow is read and executed by the CPU 30. The RAM 34 is used as a work area during the execution of various programs by the CPU 30 and the like.

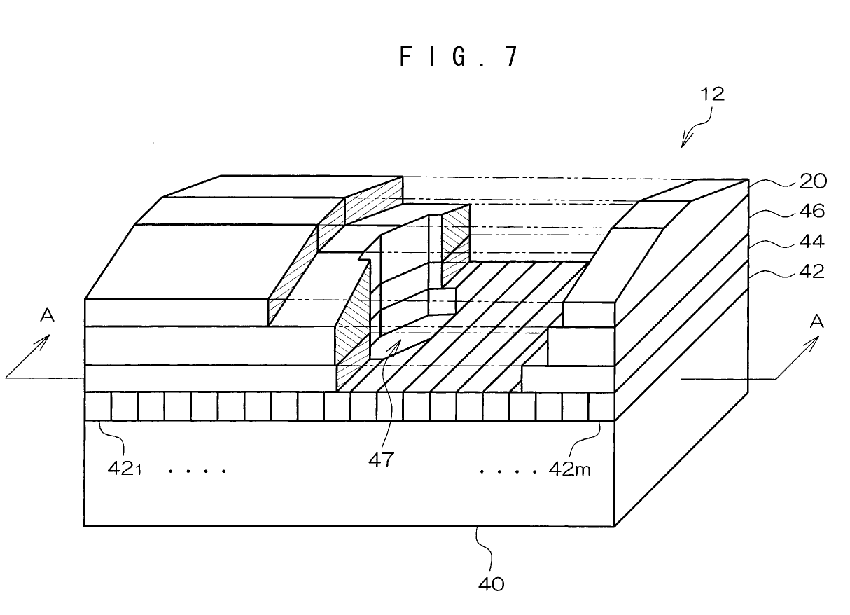

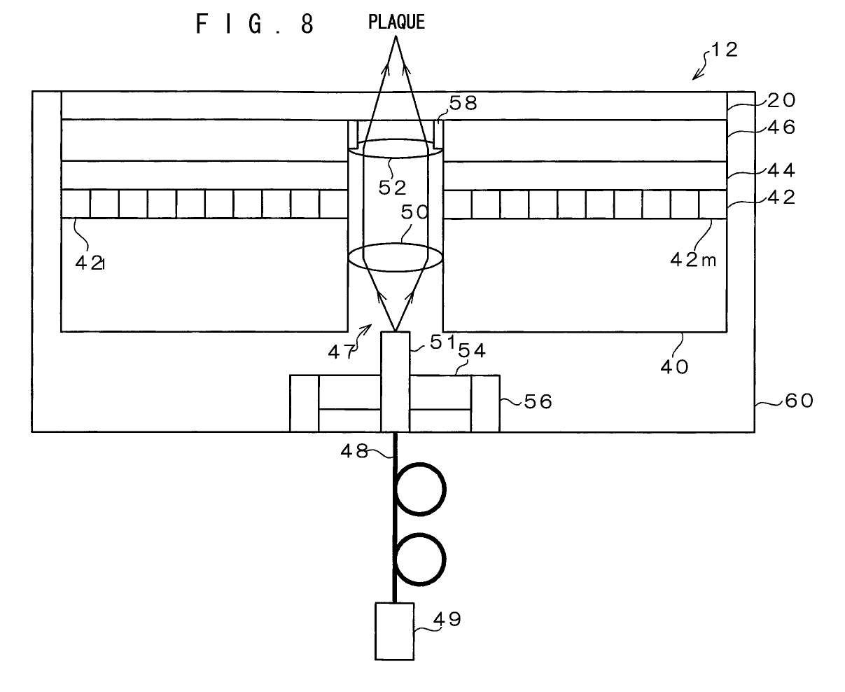

Next, an example of a specific structure of the ultrasound probe 12 of the present exemplary embodiment will be described in detail referring to FIG. 7 and FIG. 8.

FIG. 7 is a schematic view of a portion of the example of the ultrasound probe 12 relating to the present exemplary embodiment. The ultrasound probe 12 of the present exemplary embodiment is constituted to include a stacked body in which a backing material 40, a piezoelectric element 42, an acoustic matching layer 44, an acoustic lens 46 and the cooling portion 20 are sequentially stacked. At a central portion of the ultrasound probe 12, a guide hole 47 for the laser light to pass through is formed through the backing material 40, the piezoelectric element 42, the acoustic matching layer 44 and the acoustic lens 46. In the present exemplary embodiment, the piezoelectric element 42, the acoustic matching layer 44 and the acoustic lens 46 correspond to the image acquisition section 14.

The backing material 40 is for absorbing, of ultrasound generated by the piezoelectric element, ultrasound that is directed rearward (the direction away from the human body) and suppressing excess vibrations of the piezoelectric element. As a specific example, a rubber or the like may be mentioned. The piezoelectric element 42 is for generating the ultrasound and monitoring the ultrasound that is reflected from internal portions of the living body. In the present exemplary embodiment, the piezoelectric element 42 is structured by short strip-form piezoelectric elements, m in number, to which respective electrodes (not illustrated) are connected. However, the piezoelectric element 42 is not limited thus and may be structured by plural piezoelectric elements in the form of an array. The acoustic matching layer 44 is for attaining matching between acoustic impedances of the piezoelectric element and the human body and effectively transmitting the ultrasound into the living body. The acoustic lens 46 is for refracting and constricting the ultrasound. As a specific example, a silicone rubber or the like may be mentioned.

In the present exemplary embodiment, the cooling portion 20 is formed such that water flows through a supply portion (not illustrated) between a pair of transparent films. A temperature of the cooling water is set to be low. Thus, a cooling unit is cooled. The transparent members are not limited to films, and are not particularly limited so long as they do not result in high absorption of (i.e., they transmit) laser light and ultrasound, such as glass, plastic or the like. Furthermore, the medium that is supplied for cooling is not limited to cooling water, and is not particularly limited as long as it is a medium that does not result in high absorption of laser light or ultrasound, such as another liquid, nitrogen gas obtained from liquid nitrogen, or the like.

FIG. 8 illustrates a sectional view of the whole of the ultrasound probe 12 when the ultrasound probe 12 is sectioned along A-A and viewed in the direction of the arrows. The ultrasound probe 12 of the present exemplary embodiment is constituted with the stacked body illustrated in FIG. 7 (described above), a collimator lens 50, a focusing lens 52, a focusing lens variation piezoelectric element 58, a ferrule 51, ferrule variation piezoelectric elements 54 and ferrule variation piezoelectric elements 56 accommodated inside a housing 60. Herein, the collimator lens 50, the focusing lens 52 and the focusing lens variation piezoelectric element 58 of the present exemplary embodiment are the focusing section 16, and the focusing lens variation piezoelectric element 58 is the focusing distance variation section 18. The ferrule 51, the ferrule variation piezoelectric elements 54, the ferrule variation piezoelectric elements 56, an optical fiber 48 and a laser module 49 are the laser light irradiation section 24, and the laser module 49 is the power output alteration section 26.

Laser light with a wavelength from 1201 nm to 1227 nm, which is guided from the laser module 49 by the optical fiber 48, is made parallel at the collimator lens 50. This parallel light passes through the focusing lens 52, and is irradiated so as to be focused through the body surface onto the living body interior (a plaque). Herein, the irradiation of laser light is mainly implemented by pulse driving. This is because heat is transferred to the surroundings of an irradiation location if continuous light is irradiated at a living body, and the possibility of causing heat damage to regular tissues outside the plaque is to be suppressed.

When the focusing position is to be adjusted in the depth direction (adjusting the position of the focusing point), a voltage is applied to the focusing lens variation piezoelectric element 58 and the focusing lens 52 is moved in the optical axis direction of the focusing lens 52. When adjusting in a direction parallel to the body surface (a direction orthogonal to the optical axis direction), voltage is applied to the ferrule variation piezoelectric element 54 and/or the ferrule variation piezoelectric elements 56 and the ferrule 51 of the optical fiber 48 is moved in the direction parallel to the body surface. Thus, the laser light may be focused at arbitrary positions in three dimensions and the laser light may be selectively irradiated.

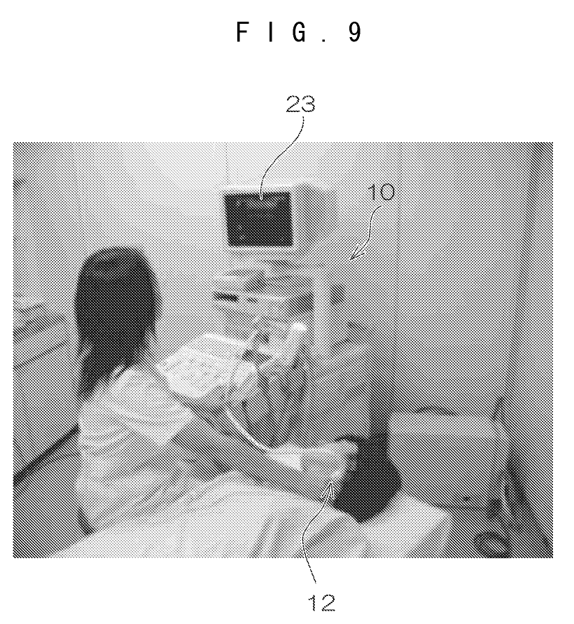

Next, a treatment method with the laser treatment device 10 of the present exemplary embodiment will be described in detail with reference to FIG. 9 to FIG. 11.

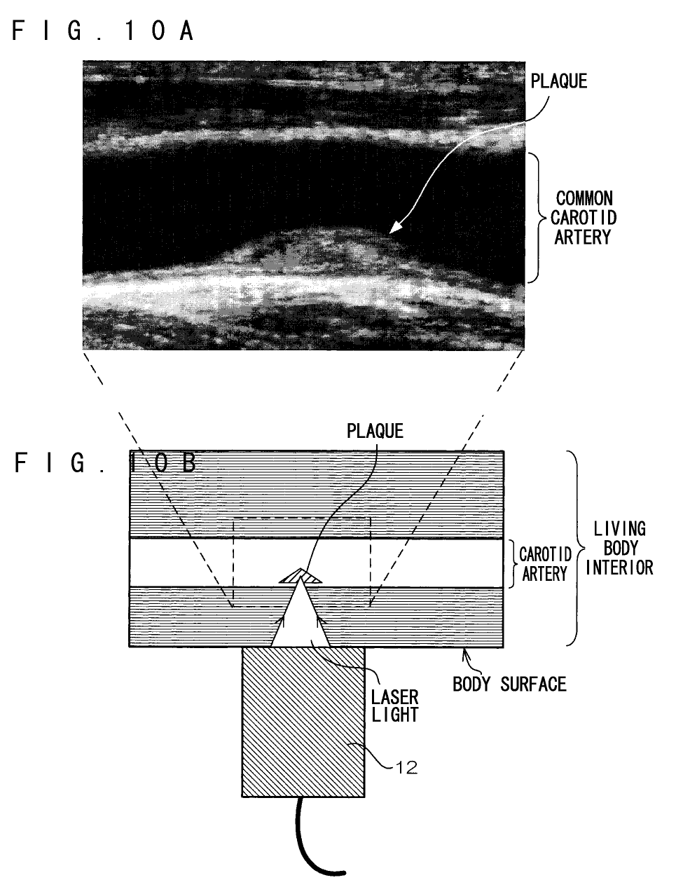

As shown in FIG. 9, with the laser treatment device 10 of the present exemplary embodiment, the form of a carotid artery is diagnosed at the display section 23 while the ultrasound probe 12 is pressed against the neck area of a patient (a region at which plaques are to be checked for). When a plaque is identified by the diagnosis, as illustrated in FIG. 10A, laser light is irradiated from the ultrasound probe 12 at the plaque, as shown in FIG. 10B. The laser light irradiated from the ultrasound probe 12 enters into the body of the patient from outside the body, and is irradiated at the plaque inside the carotid artery. The irradiated laser light is absorbed by the plaque and heats the plaque. Hence, lipid components in the plaque are dissolved into the blood and/or apoptosis/necrosis of the fat cells is caused. In the present exemplary embodiment, the laser light is irradiated by a doctor directing the irradiation with a control section (not illustrated) of switches and the like. The doctor directs the irradiation of laser light (irradiation durations) while monitoring the condition of the plaque.

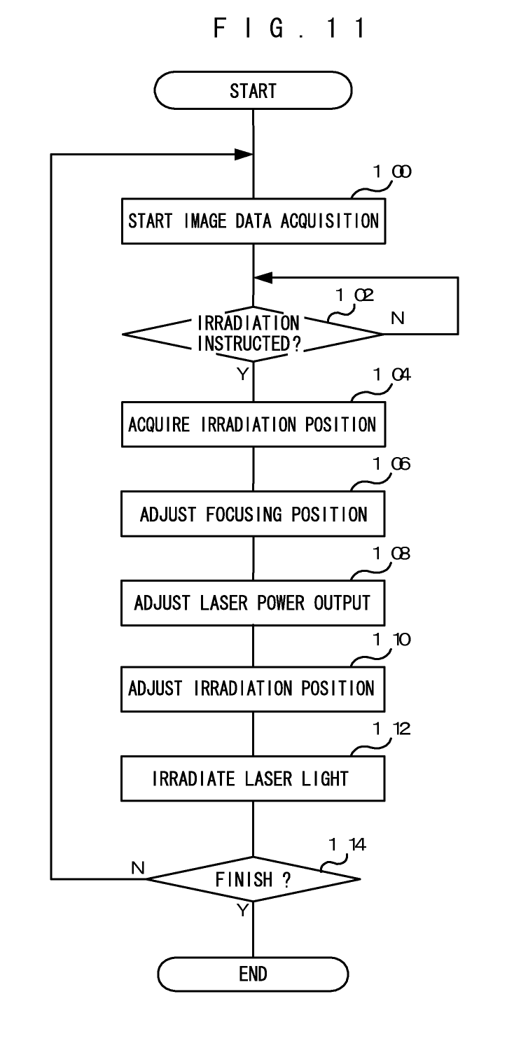

FIG. 11 shows a flowchart of an example of the treatment method of the laser treatment device 10 of the present exemplary embodiment. The flowchart shown in FIG. 11 is executed, for example, when a power supply to the laser treatment device 10 is turned on, when an instruction is inputted by a doctor or the like, or the like.

Firstly, in step 100, acquisition by the image acquisition section 14 of image data for an image of the interior of a body of a patient is commenced. Herein, the acquired image data is displayed at the display section 23. Hence, the doctor judges whether there is a plaque or not. In step 102, it is judged whether or not there is an instruction to irradiate laser light. When there is no instruction, this judgement is negative and the processing is in a standby state. Then, when there is an instruction, the judgement is positive and the processing advances to step 104. In the present exemplary embodiment, cooling of the body surface of the patient (a body surface region at which laser light will be irradiated) by the cooling portion 20 begins when there is an instruction to irradiate laser light.

In step 104, the position of a plaque is acquired with the display section 23, and a progression distance of laser light to the plaque is calculated. Then, in step 106, on the basis of the acquired plaque position (the calculated laser light progression distance), the focusing position of the focusing lens is adjusted (described in detail hereafter) by the focusing distance variation section 18 such that the laser light will be focused at the position of the plaque.

Then, in step 108, the power output (light intensity) of the laser light to be irradiated is adjusted. The laser light power output is calculated on the basis of the acquired plaque position (the calculated laser light progression distance), and the power output alteration section 26 is instructed to set to the calculated power output.

In step 110, the position to be irradiated with the laser by the laser light irradiation section 24 is adjusted in accordance with the position, acquired by the plaque position acquisition section 22, of the plaque in the directions parallel to the body surface.

Next, in step 112, laser light is irradiated onto the plaque from the laser light irradiation section 24. In the present exemplary embodiment, the laser light continues to be irradiated for as long as instructed by the doctor as mentioned earlier. While the laser light continues to be irradiated, the irradiation position may be acquired and adjustment of the focusing position, adjustment of the laser power output and the like may be implemented. When the irradiation of laser light finishes, the processing advances to step 114.

In step 114, it is judged whether or not the present processing is to end. If the judgement is negative, the present processing returns to step 100 and is repeated. On the other hand, if there is an instruction from the doctor to finish, or if the power supply of the laser treatment device 10 is turned off or the like, the acquisition of image data ends and the present processing ends.

As has been described hereabove, the ultrasound probe 12 of the present exemplary embodiment acquires the position of a plaque with the plaque position acquisition section 22 on the basis of image data acquired by the image acquisition section 14, and irradiates laser light with a wavelength from 1201 nm to 1227 nm, which is guided by the optical fiber 48 from the laser module 49, such that the laser light passes through the focusing lens 52 and is focused through the body surface onto the position of the plaque inside the living body.

Therefore, laser light with a wavelength that has little effect on the human body and affects only lipid components may be irradiated and, because a light density at the body surface may be reduced, it is possible to prevent the body surface at which the laser light is irradiated being subjected to burn damage, pain or the like, and body tissues around the plaque being damaged. Therefore, the laser may be irradiated at lipid components in the living body from outside the body, and treatment of peripheral arteriosclerosis in the neck, hands, feet or the like may be performed with a low level of invasiveness into the human body.

Moreover, because the focusing position may be adjusted in three dimensions, the depth direction and the directions parallel to the body surface, it is simple to selectively attack the plaque alone, and a high level of treatment effectiveness may be obtained.

Further yet, effects may be provided in that: because the body is not cut open, invasiveness is low and recovery after treatment is rapid; because it is possible to control treatment effectiveness by controlling the power output of laser light, the possibility of infarctions resulting may be reduced; treatment may be repeated many times over a lifetime; the possibility of damage to blood vessels is low; and the like. This improves QOL (quality of life), contributes to restraint of treatment expenses, and so forth.

### Second Exemplary Embodiment

Herebelow, another specific example of an exemplary embodiment of the present invention will be described in detail with reference to the drawings. Here, a laser treatment device of the present exemplary embodiment has substantially the same constitution and effects as the ultrasound probe 12 of the first exemplary embodiment, and differs only in the ultrasound probe and the laser light irradiation section. Accordingly, portions that are the same are assigned the same reference numerals and will not be described, and only portions that differ will be described in detail. The treatment method too is substantially the same as in the first exemplary embodiment, so will not be described.

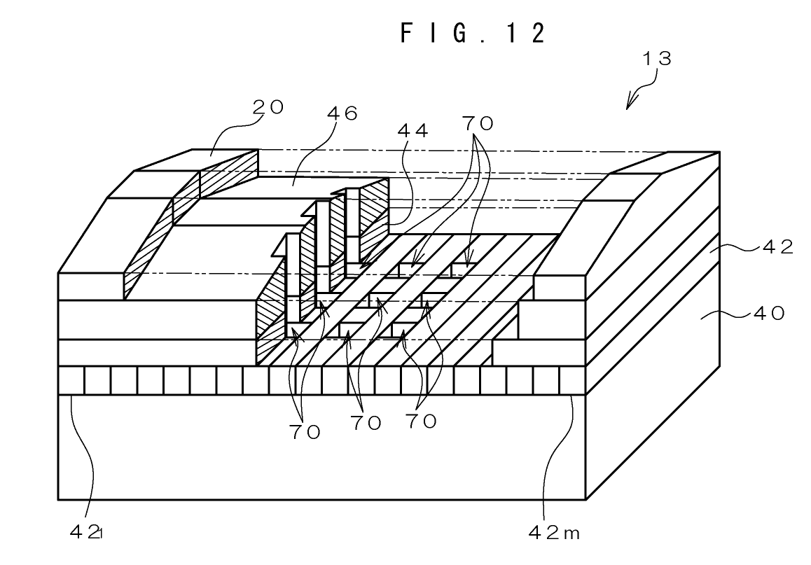

FIG. 12 is a schematic view of a portion of an example of an ultrasound probe 13 relating to the present exemplary embodiment. In the ultrasound probe 13 of the present exemplary embodiment, plural guide holes 70 (in a number of laser lights to be irradiated) for the laser lights to pass along are formed in the backing material 40, the piezoelectric element 42, the acoustic matching layer 44 and the acoustic lens 46.

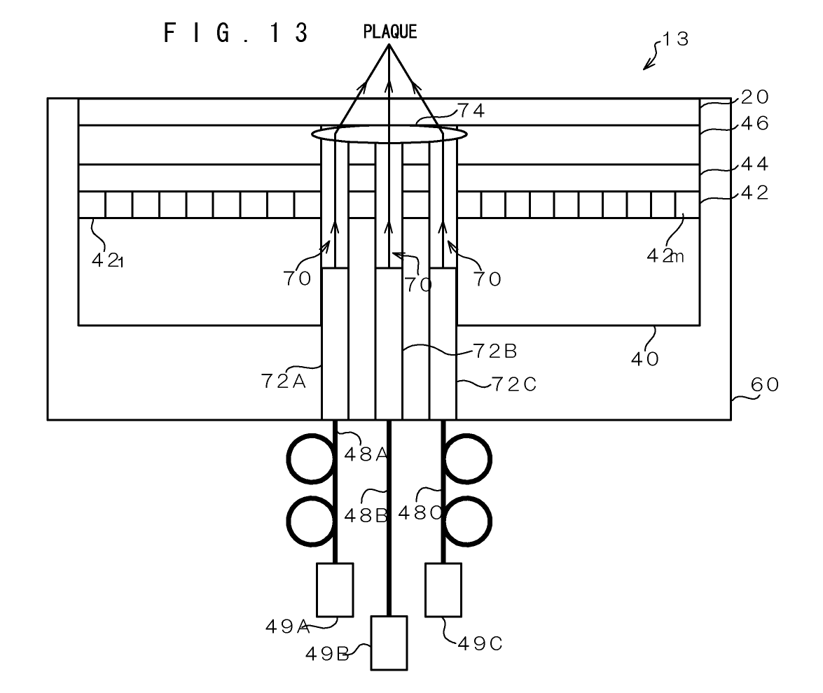

FIG. 13 shows a sectional view of the whole of the ultrasound probe 13 when the ultrasound probe 13 illustrated in FIG. 12 is sectioned along A-A and viewed in the direction of the arrows. The ultrasound probe 13 of the present exemplary embodiment is constituted with a condensing variable focus point lens (a liquid lens) 74 and plural ferrules 72 (the ferrules 72A, 72B and 72C in FIG. 13) accommodated inside the housing 60.

Laser lights (parallel lights) with wavelengths from 1201 nm to 1227 nm, which are guided from a plurality of the laser module 49 (laser modules 49A, 49B and 49C) by a plurality of the optical fiber 48 (optical fibers 48A, 48B and 48C), pass through the liquid lens 74, and are irradiated so as to be focused through the body surface at the living body interior (a plaque).

Graded fibres with the function of a collimator lens are fused to emission side distal ends of the optical fibers 48 of the present exemplary embodiment. Thus, the laser lights from the emission ends become parallel lights.

When the focusing position is to be adjusted in the depth direction (adjusting the position of the focusing point), a voltage is applied to the liquid lens 74 and the focusing position is altered by the shape of the lens being altered.

As described above, in the present exemplary embodiment, plural laser lights may be irradiated at a plaque. Therefore, this is more suitable for cases requiring high power output, multiples of power output may be provided by increasing the number of laser modules 49, and the scope of treatment may be broadened.

Moreover, because the guide holes 70 for passing the laser lights may be made small, ultrasound images may be acquired more precisely.

In the first exemplary embodiment and the second exemplary embodiment described above, cases in which the image acquisition section 14 acquires an image with ultrasound (an ultrasound probe) have been described in detail, but these are not limiting and other alternative technologies may be employed. For example, optical coherence tomography (OCT), a confocal microscope that uses infrared laser light, with which deep positions are not visible, and the like may be employed.

Further, in the first exemplary embodiment and second exemplary embodiment described above, the cooling portion 20 is provided so as to cover the whole of a surface portion at which the ultrasound probe touches against the human body. This is not limiting, and the cooling portion 20 may be formed so as to cover just a portion at which laser light is irradiated and surrounding portions. However, it is preferable to acquire an ultrasound image with a surface that touches against the human body being flat.

The focusing lens variation piezoelectric element 58 is employed in the above-described first exemplary embodiment and the liquid lens 74 is employed in the second exemplary embodiment, but these are not limiting. Which of the lenses is to be employed may be selected in accordance with other design points and the like of the ultrasound probe, and desired characteristics and the like.

In the above-described first exemplary embodiment and second exemplary embodiment, cases are described in which plaques inside the human body are treated, but this is not limiting. Employment for organisms other than humans (animals and the like) is also possible. Employment is further possible in treatments of obesity (weight reduction), by irradiating subdermal fats besides plaques and stimulating the metabolism. In any case, the treatment may be performed with low invasiveness into the living body.

What is claimed is:

1. A laser treatment device comprising:
an image acquisition section that acquires an image of a treatment region including a lipid component inside a living body and surroundings of the treatment region;
a position acquisition section that acquires a position of the treatment region in an optical axis direction of laser light, on the basis of image data of the image acquired by the image acquisition section;
an irradiation section that irradiates laser light with a wavelength from 1201 nm to 1227 nm at the treatment region from outside the body; and
a focusing section that focuses the laser light irradiated from the irradiation section at the position of the treatment region acquired by the position acquisition section.

2. The laser treatment device according to claim 1, wherein
the irradiation section includes a light intensity alteration section that alters a light intensity of the irradiated laser light, and
the focusing section includes a focusing lens and a focusing distance variation section that alters a focusing distance of the focusing lens,
and wherein the laser treatment device further includes
a control section that controls the light intensity alteration section and the focusing distance variation section in accordance with the position of the treatment region such that the light intensity of the laser light is a light intensity capable of treating the treatment region.

3. The laser treatment device according to claim 2, wherein the focusing distance variation section comprises a movement portion that moves the focusing lens in the optical axis direction of the laser light.

4. The laser treatment device according to claim 2, wherein the focusing lens comprises a liquid lens whose focusing distance is alterable.

5. The laser treatment device according to claim 1, wherein a plurality of the irradiation sections is provided, and
the focusing section focuses the plurality of laser light irradiated from the plurality of irradiation sections.

6. The laser treatment device according to claim 1, further comprising a display section that displays the image of the treatment region and the surroundings of the treatment region acquired by the image acquisition section.

7. The laser treatment device according to claim 1, further comprising a cooling portion that cools a surface of the living body at which the laser light irradiated from the irradiation section is irradiated.

8. The laser treatment device according to claim 7, wherein the cooling section includes a pair of thin-film members that transmit the laser light, and a supply portion, between the pair of thin-film members, that supplies a liquid or gas for cooling the living body.

9. The laser treatment device according to claim 1, wherein the image acquisition section comprises an ultrasound image acquisition section that includes an ultrasound probe and that acquires an ultrasound image on the basis of reflected waves of ultrasound, which is outputted from the ultrasound probe toward the interior of the living body from outside the body.

10. The laser treatment device according to claim 1, wherein a numerical aperture of the focusing section is a numerical aperture such that a beam area of the laser light at a surface of the living body is at least 1.7 times a beam area of the laser light focused at the treatment region.
